(19) United States
(12) Patent Application Publication
(10) Pub. No.: US 2005/0244916 A1
(43) Pub. Date: Nov. 3, 2005

(54) Methods for predicting protein activity based on identification of multidimensional signatures

(76) Inventors: Michael Yeaman, Redondo Beach, CA (US); Nannette Yount, San Juan Capistrano, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/087,424

(22) Filed: Mar. 22, 2005

Related U.S. Application Data
(60) Provisional application No. 60/555,437, filed on Mar. 22, 2004.

(51) Int. Cl. G06F 19/00; G01N 33/48; G01N 33/50; C12Q 1/18

(52) U.S. Cl. 435/32; 702/19

(57) ABSTRACT

The present invention provides a method for predicting antimicrobial activity of a candidate protein by determining the correlation between a multidimensional antimicrobial signature and a multidimensional antimicrobial signature model. The invention also is directed to a method for identifying a protein having antimicrobial activity by screening a library of candidate proteins to identify a multidimensional antimicrobial signature. Also provided by the invention is a method for improving the antimicrobial activity of a protein by altering the multidimensional antimicrobial signature of the protein to increase the similarity to a multidimensional antimicrobial signature model.

### BACKGROUND OF THE INVENTION

This application claims the benefit of priority of U.S. Provisional application Ser. No. 60/555,437, filed Mar. 22, 2004, of which the entire contents is incorporated herein by reference.

This invention relates to experimental proteomics and, more specifically to methods for predicting protein activity based on identification of multidimensional signatures.

Nature provides a context in which organisms across the phylogenetic spectrum are confronted by potential microbial pathogens. In turn, natural selection provides a corresponding requirement for rapid and effective molecular stratagems of host defense against unfavorable microbial infection. Antimicrobial peptides represent a key result of this co-evolutionary relationship. While higher organisms have evolved complex and adaptive immune systems, virtually all organisms rely upon primary innate immune mechanisms that are rapidly deployed to ward off microbial invasion. Discoveries over the last decade indicate that antimicrobial peptides elaborated by essentially all organisms play integral roles in these innate mechanisms of antimicrobial host defense.

Antimicrobial peptides may be generally categorized as those with or without disulfide bridges. Those that contain disulfides commonly adopt β-sheet structures, while those lacking cysteine crosslinkages often exhibit α-helical conformation. Antimicrobial peptides from both classes have a number of conserved features that likely contribute to their toxicity to microorganisms, including: 1) small size, typically ranging from 12-50 amino acids; 2) cationicity, with net charges ranging from +2 to +7 at pH 7; and 3) amphipathic stereogeometry conferring relatively polarized hydrophilic and hydrophobic facets (Yeaman and Yount, Pharmacol. Rev. 55:27 (2003)). The limited size of these polypeptides places restrictions on the structural repertoire available to meet these requirements. Despite these limitations, as a group antimicrobial peptides display a high degree of variability at non-conserved sites, with amino acid substitution rates on the order of those associated with positive selection (A. L. Hughes, Cell. Mol. Life Sci. 56:94 (1999)). These observations are consistent with the hypothesis that co-evolutionary selective pressures drive host-pathogen interactions (M. J. Blaser, N. Engl. J. Med. 346:2083 (2002)).

Amino acid sequence motifs have previously been identified within certain antimicrobial peptide subclasses (eg., the cysteine array in certain mammalian defensins; White et al., Curr. Opin. Struct. Biol. 5:521 (1995)). Yet, comparatively little is known about more comprehensive relationships uniting all antimicrobial peptides. Conventional sequence analyses performed have yielded limited sequence conservation, and no universal structural homology has been identified amongst antimicrobial peptides. If present, such a consensus motif across the diverse families of antimicrobial peptides would provide insights into the mechanism of action of these molecules, yield information on the evolutionary origin of these sequences, and allow prediction of antimicrobial activity in molecules recognized to have other functions.

Thus, there exists a need for employing multidimensional proteomic techniques to determine structural commonalities amongst peptides elaborated in phylogenetically diverse organisms—microbial to human—and explore the potential convergence of structural paradigms in these molecules. The present invention satisfies this need and provides related advantages as well.

### SUMMARY OF THE INVENTION

The present invention provides a method for predicting antimicrobial activity of a candidate protein by determining the correlation between a multidimensional antimicrobial signature and a multidimensional antimicrobial signature model. The invention also is directed to a method for identifying a protein having antimicrobial activity by screening a library of candidate proteins to identify a multidimensional antimicrobial signature. Also provided by the invention is a method for improving the antimicrobial activity of a protein by altering the multidimensional antimicrobial signature of the protein to increase the similarity to a multidimensional antimicrobial signature model.

### BRIEF DESCRIPTION OF THE DRAWINGS

FIG. 1 shows conventional antimicrobial peptide structure classification and distribution. Relationship amongst structure and predominance is summarized for the commonly recognized antimicrobial peptide classes. Concatenation represents the proportionate distribution of peptides encompassing a given structural class, as calculated from the Antimicrobial Sequences Database. Numbers of peptides classified in each group are indicated in brackets for each class.

FIG. 2(A) shows multiple sequence alignment of antimicrobial peptides examined. The MSA of the β-sheet peptide study set was generated using the Clustal W tool (Version 1.81; Higgins and Sharp, Gene 73:237 (1988); Higgins and Sharp, Comput. Appl. Biosci. 5:151 (1989)), as visualized with Jalview (M. Clamp, Jalview—java multiple alignment editor, version 1.7b (1998). Public domain (www.ebi.ac.uk/jalview/)). The coloration scheme is formatted to the Clustal degree of conservation. Individual peptides are designated by the following information series: peptide name, (source genus), and [Swiss Protein accession code]: protegrin 1, (Sus), [3212589]; gomesin, (Acanthoscurria), [20664097]; drosomycin, (Drosophila), [2780893]; MGD-1, (Mytilus), [12084380]; tachyplesin I, (Tachypleus), [84665]; mytilin A, (Mytilus), [6225740]; sapecin, (Sarcophaga), [20151208]; HNP-3, (Homo), [229858]; Ah-Amp1, (Aesculus), [6730111]; AFP-1, (Aspergillus), [1421258]; mBD-8, (Mus), [15826276]; thanatin, (Podisus), [6730068]; and gaegurin-1, (Rana), [1169813].

FIG. 2(B) shows convergence in the sequence patterns of cysteine-containing antimicrobial peptides. The consensus primary structural motifs were identified amongst the prototypical disulfide-containing antimicrobial peptide study set. Sequence data and disulfide arrays indicated were derived from the following sources (in descending order): α-defensins (Yount et al., J. Biol. Chem. 274:26249 (1999)); β-defensins (Yount et al., J. Biol. Chem. 274:26249 (1999)); insect defensins (sapecin; Hanzawa et al., FEBS Lett. 269:413 (1990)); insect CS-αβ peptides (drosomycin; Landon et al., Protein Sci. 6:1878 (1997)); plant CS-αβ peptides (Ah-AMP-1; Fant et al., Proteins 37:388 (1999)); crustacea CS-αβ peptides (MGD-1; Yang et al., Biochemistry 39:14436 (2000)); gaegurin (gaegurin-1; Park et al., Biochem. Biophys. Res. Commun. 205:948 (1994)); protegrin (protegrin-1; Fahrner et al., Chem. Biol. 3:543 (1996)); gomesin (Silva et al., J. Biol. Chem. 275:33464 (2000); Mandard et al., Eur. J. Biochem. 269:1190 (2002)); thanatin (Mandard et al., Eur. J. Biochem. 256:404 (1998)); tachyplesin (tachyplesin I; Nakamura et al., J. Biol. Chem. 263:16709 (1988)); mytilin (mytilin Charlet et al., J. Biol. Chem. 271:21808 (1996)); AFP-1 (Campos-Olivas et al., Biochemistry 34:3009 (1995)). The primary sequences corresponding to the γ-core motif are outlined in red (see FIG. 4). Sequences are shown in their conventional dextromeric orientations (N- to C-termini from left to right) unless indicated to be projected in a levomeric orientation (levo; C- to N-termini from left to right).

FIG. 3(A-H) shows conservation of 3-dimensional signatures amongst antimicrobial peptides. Three-dimensional structural alignments were carried out by the combinatorial extension method (Shindyalov and. Bourne, Protein Eng. 11:739 (1998)), visualized using Protein Explorer (Martz, Trends Biochem. Sci. 27:107 (2002)). Comparisons are between (Ah-AMP-1 ([1BK8], Aesculus, horsechestnut tree) and (peptide name, [PDB accession code], genus, common name; RMSD): protegrin-1 ([1PG1], Sus, domestic pig; RMSD 1.2 Å; panels A and B); drosomycin ([1MYN], Drosophila, fruit fly; RMSD 1.4 Å; panels C and D); HNP-3 ([1DFN]; Homo, human; RMSD 3.2 Å; panels E and F); and magainin-2 ([2MAG]; Xenopus, frog; Gesell et al., J. Biomol. NMR 9:127 (1997); RMSD 2.6 Å; panels G and H). Respective amino- and carboxy-termini are indicated in panels A, C, E and G. Panels A, C, E, and G use the Clustal degree of 2° structure conservation coloration scheme. Panels B, D, F, and H employ the DRuMS polarity-2 color scheme, in which hydrophobic residues are colored gray, while hydrophilic residues are colored purple. By convention, cysteine residues are indicated as hydrophilic, although in these peptides, they are oxidized (cystine) and colored gray indicating hydrophobicity. Amino- (N-) and carboxy- (C-) termini for comparative peptides are denoted as N1 or N2 and C1 or C2, respective of peptides designated 1 or 2. Relative positions of the disulfide bonds are indicated as dotted yellow lines in panels A-H. See Table I for additional references. Proteins were visualized using Protein Explorer as described by Martz, Trends Biochem. Sci. 27, 107-109 (2002).

FIG. 3(I-L) demonstrates the absence of the γ-core signature in non-antimicrobial peptides. Three-dimensional conformity between prototypic antimicrobial and non-antimicrobial peptides was determined as described in FIG. 3A-H. Representative comparisons are between the antimicrobial peptide Ah-AMP1, and the following non-antimicrobial peptides (identified as formatted in FIG. 3A-H): allergen-5 ([2BBG], Ambrosia, ragweed; RMSD 6.5 Å; panel I); metallothionein II ([1AOO], Saccharomyces, yeast; RMSD 5.3 Å; panel J); TGF-α ([3TGF], Homo, human; RMSD 4.7 Å; panel K); and ferredoxin ([2FDN], Clostridium, bacterium; RMSD 7.4 Å; panel L). Each non-antimicrobial comparator peptide (blue) is shown in maximal alignment with Ah-AMP1 (gray). Amino- (N) and carboxy- (C) termini are indicated as defined in FIG. 3A-H. See Table I for references.

FIG. 4 shows conservation of they-core motif amongst disulfide-containing antimicrobial peptides. The conserved γ-core motif (red) is indicated with corresponding sequences (GXC or CXG—C motifs are denoted in red text). Examples are organized into four structural groups relative to the γ-core. Group (γ): protegrin-1, [1PG1]; gomesin [1KFP]; tachyplesin-1 [1MA2]; RTD-1 [1HVZ]; thanatin [8TFV]; hepcidin [1M4F]); Group (γ-α): sapecin [1LV4]; insect defensin A [1ICA]; heliomicin [1I2U]; drosomycin [1MYN]; MGD-1 [1FJN]; charybdotoxin [2CRD]); Group (β-γ): HNP-3 [1DFN]; RK-1 [1EWS]; BNBD-12 [1BNB]; HBD-1 [1E4S]; HBD-2 [1E4Q]; mBD-8 [1E4R]); and Group (β-γ-α): Ah-AMP-1 [1BK8]; Rs-AFP-1 [1AYJ]; Ps-Def-1 [1JKZ]; γ-1-H-thionin [1GPT]; γ-1-P-thionin [1GPS]; and brazzein [1BRZ]. Protegrin, gomesin, tachyplesin, RTD-1, and thanatin γ-core sequences (Group γ) are depicted in levomeric orientation. Other peptide data are formatted as in FIG. 3. See Table I for additional references.

FIG. 5(A-C) shows iterations of the 3-dimensional γ-core motif. Amino acid consensus patterns of the three γ-core sequence isoforms are shown. Coloration represents the most common residue (>50% frequency) at a given position, as adapted from the RASMOL schema: cysteine (C), yellow; glycine (G), orange; lysine or arginine, royal blue; serine or threonine, peach; leucine, isoleucine, alanine or valine, dark green; aromatic, aqua; and variable positions (<50% consensus), gray.

FIG. 6(A-I) shows molecules exemplifying structure-based or activity-based validation of the multidimensional signature model. Representative molecules retrieved using the enantiomeric sequence patterns were identified (Table II) and analyzed for presence or absence of a γ-core motif as described. Thus, appropriate molecules were identified to challenge each of the respective model-based predictions. Three-dimensional structures visualized using Protein Explorer are indicated for: brazzein ([1BRZ], Pentadiplandra, J'Oblie berry, panels A, D, and G; Caldwell et al., Nat. Struct. Biol. 5:427 (1998)); charybdotoxin ([2CRD], Leiurus, scorpion, panels B, E, and H; Bontems et al,. Biochemistry 31:7756 (1992)), tachyplesin I ([1MA2], Tachypleus, horseshoe crab, panels C, F, and I); and metallothionein II (see FIG. 3). As in FIG. 3, comparative panels A-C use the Clustal degree of 2° structure conservation coloration scheme. Panels D-F employ the DRuMS polarity-2 color scheme, in which hydrophobic residues are colored gray, and hydrophilic residues are colored purple. As in FIG. 4, amino acids comprising the γ-core motifs are highlighted in red within the 3-dimensional structures of these representative peptides. Other data are formatted as in FIG. 3.

FIG. 7 shows experimental validation of the predictive accuracy of the multidimensional signature model. Standard radial diffusion assays were conducted using 10 μg of specified peptide: defensin HNP-1 (HNP); brazzein (BRZ); charybdotoxin (CTX); or metallothionein II (MTL). Recombinant brazzein reflecting the published 3-dimensional structure (1BRZ) as determined by nuclear magnetic resonance spectroscopy was kindly provided by Drs. J. L. Markley and F. M. Assadi-Porter, the University of Wisconsin 25. Charybdotoxin, metallothionein II, and defensin HNP-1 were obtained from commercial sources. Antimicrobial activity was assessed using a well-established solid-phase diffusion method as described by Tang et al., Infect. Immun. 70: 6524-6533 (2002). Assays included well characterized organisms: Staphylococcus aureus (ATCC 27217, Gram-positive coccus); Bacillus subtilis (ATCC 6633, Gram-positive bacillus); Escherichia coli (strain ML-35, Gram-negative bacillus); and Candida albicans (ATCC 36082, fungus). In brief, organisms were cultured to logarithmic phase and inoculated (106 colony forming units/ml) into buffered molecular-biology grade agarose at the indicated pH. Peptides resuspended in sterile deionized water were introduced into wells formed in the underlay, and incubated for 3 h at 37° C. Nutrient-containing overlay medium was then applied, and assays incubated at 37° C. or 30° C. for bacteria or fungi, respectively. After 24 h, zones of complete or partial inhibition were measured. All assays were repeated independently a minimum of two times at pH 5.5 (panels A-D) or pH 7.5 (panels E-H) to assess the influences of pH on peptide antimicrobial activities versus microorganisms. Histograms express mean (±standard deviation) zones of complete (blue) or incomplete (yellow) inhibition of growth. These data establish the direct antimicrobial activities of brazzein and charybdotoxin. Metallothionein II lacked antimicrobial activity under any condition assayed. Note differences in scale.

FIG. 8(A) shows phylogenetic relationship amongst structural signatures in prototypical antimicrobial peptides. Relative evolutionary distances are indicated at branch nodes in this average distance dendrogram (Saito and Nei, Mol. Biol. Evol. 4:406 (1987)). Representative peptides for which structures have been determined are (descending order): AFP (AFP-1; Aspergillus, fungal); PRG1 (Protegrin-1; Sus, domestic pig); GOME (Gomesin; Acanthoscurria, spider); THAN (Thanatin; Podisus, soldier bug); HNP3 (Human neutrophil peptide-3; Homo, human); MGDI (MGD-1; Mytilus, mussel); SAPE (Sapecin; Sarcophaga, flesh fly); MBD8 (Murine β-defensin-8; Mus, mouse); DMYN (Drosomycin; Drosophila, fruit fly); Ah-AMP1 (AMP-1; Aesculus, horsechestnut tree). Color schema are the Clustal degree of 2° structure conservation. These data illustrate the concept that the γ-core is the common structural element in these peptides, suggesting it is an archetype motif of the antimicrobial peptide signature (see FIG. 4).

FIG. 8(B) shows modular iterations of multidimensional signatures in disulfide-stabilized antimicrobial peptides. Distinct configurations integrating the γ-core are found in naturally occurring antimicrobial peptides from diverse organisms. Specific examples are used to illustrate this theme (modular formulae are as described in the text): [γ], Protegrin-1; [γα₁], MGD-1; [γβ₁], HNP-3; and [γα₁β₁], Ah-AMP-1. Color schema and peptide identification are as indicated in FIG. 3(A, C, E, G).

FIG. 9 shows conservation of the multidimensional signature in disulfide-containing antimicrobial peptides. This triple alignment demonstrates the dramatic 3-dimensional conservation in antimicrobial peptides from phylogenetically diverse species spanning 2.6 billion years of evolution: fruit fly (Drosophila; [1MYN]), mussel (Mytilus; [1FJN]) and horsechestnut tree (Aesculus; [1BK8]). The striking degree of 3-dimensional preservation reflects a unifying structural code amongst these broad classes of disulfide containing host defense effector molecules. Alignment was carried out using the Vector Alignment Search Tool (VAST) available through the National Center for Biotechnology Information (NCBI). Secondary structure is indicated by the CN3D coloration schema: sheet, gold; helix, green; turn/extended, blue.

FIG. 10, panels A-E, depict amino acid sequences of γ-core signature motifs amongst disulfide-containing antimicrobial peptides. Nomenclature and coloration are as indicated in FIGS. 2 and 3 of the primary manuscript; standard abbreviations are used for peptide names where appropriate. Lavender shading of molecule identities in Groups IID and IIIB indicates peptides aligned in the levomeric orientation. These sequences correspond to the γ-core pattern map as depicted in FIG. 3 of the primary manuscript.

FIG. 11, Panels A and B, show peptides with predicted antimicrobial activity based on the multidimensional signature. Candidate peptides were identified by VAST alignment, 3D-RMSD, and manual comparisons; all RMSD scores compared with Ah-AMP-1 (1BK8; Aesculus); threshold typically >4.5 excluded; each sequence is identified by NCBI accession number.

### DETAILED DESCRIPTION OF THE INVENTION

This application file contains drawings executed in color. Copies of this patent or application publication with color drawings will be provided by the Office upon request and payment of the necessary fee.

This invention is directed to methods for identifying multidimensional protein signatures that are useful as predictors of protein activity. Prior to this invention it was unknown that proteins can be classified based on common multidimensional signatures that are predictive of activity. While exemplified herein for a subclass of antimicrobial peptides, this discovery allows for the invention methods of using experimental proteomics techniques to identify multidimensional protein signatures that are predictive of protein activity.

Based, in part, on the discovery of structural signatures in antimicrobial peptides, the invention provides methods for designing, creating or improving anti-infective agents and anti-infective strategies that are refractory to microbial resistance. The invention methods can improve the efficacy of a drug or a drug candidate by altering the multidimensional antimicrobial signature so as to approximate the multidimensional signature model.

In one embodiment, the invention provides a method for predicting antimicrobial activity of a candidate protein by determining the presence a multidimensional antimicrobial signature in a candidate protein, and comparing the multidimensional antimicrobial signature to a multidimensional antimicrobial signature model. As taught herein, the degree of similarity between the multidimensional antimicrobial signature of the candidate protein and the multidimensional antimicrobial signature model is predictive of antimicrobial activity of the candidate protein.

In a further embodiment, the invention provides a method for identifying a protein having antimicrobial activity by screening a library of candidate proteins to identify a multidimensional antimicrobial signature in a candidate protein, and subsequently comparing the multidimensional antimicrobial signature to a multidimensional antimicrobial signature model. As taught herein, the degree of similarity between the multidimensional antimicrobial signature of the candidate protein and the multidimensional antimicrobial signature model is predictive of antimicrobial activity of the candidate protein.

In a further embodiment, the invention provides a method for improving the antimicrobial activity of a protein by altering the multidimensional antimicrobial signature of the protein to increase the degree of similarity between the multidimensional antimicrobial signature of the protein and a multidimensional antimicrobial signature model. The invention also provides a protein having improved antimicrobial activity as a result of alteration of the multidimensional antimicrobial signature of the protein to increase the degree of similarity between the multidimensional antimicrobial signature of the protein and a multidimensional antimicrobial signature model.

In a further embodiment, the invention provides a method for designing a protein having antimicrobial activity by incorporating configurations that include iterations of a γ-core signature into a peptide structure that is designed. The invention also provides a protein having antimicrobial activity designed by incorporating configurations that include iterations of a γ-core signature into a peptide structure.

As used herein, the term “multidimensional protein signature” is intended to refer to a set of essential structural components that make up a structural motif characteristic of a class or subclass of proteins. A multidimensional protein signature can incorporate any structural information ascertainable, including, information regarding primary structure, including amino acid sequence, composition, and distribution patterns; secondary structure, stereospecific sequence and 3-dimensional conformation. As used herein, the term “multidimensional protein signature model” refers to a protein that represents the essential structural components associated with a particular multidimensional protein signature. Individual peptides each contain an iteration of the multidimensional signature, and the essential features of this signature are reflected in the multidimensional signature model.

As used herein, the terms “gamma-core motif,” “γ-core,” “γ-core signature” and equivalents thereof refer to a multidimensional protein signature, in particular a multidimensional antimicrobial signature, that is characterized by two anti-parallel 1-sheets interposed by a short turn region with a conserved GXC (dextromeric) or CXG (levomeric) sequence pattern integrated into one 1-sheet. Additional features that characterize the γ-core motif include a hydrophobic bias toward the C-terminal aspect and cationic charge positioned at the inflection point and termini of the 1-sheet domains, polarizing charge along the longitudinal axis of the γ-core.

As used herein, the term “protein activity” is intended to mean a functional activity or bioactivity of a protein.

Many disulfide-containing antimicrobial peptides have multiple structural domains that encompass β-sheet and/or α-helical motifs connected through an interposing region. As described herein, the invention methods provide a strategy incorporating a synthesis of proteomic and experimental methods to identify essential structural features integral to antimicrobial bioactivity that are shared amongst broad classes of antimicrobial peptides. Stereospecific sequence and 3-dimensional conformation analyses of cysteine-containing antimicrobial peptides with known structures were integrated and reduced to identify essential structural components. These approaches enabled the identification of sequence patterns and a 3-dimensional conformation integral to a multidimensional signature common to virtually all non-cyclic antimicrobial peptides containing disulfide bridges. This compelling signature transcends class-specific motifs identified previously, and reflects a unifying structural code in antimicrobial peptides from organisms separated by profound evolutionary distances.

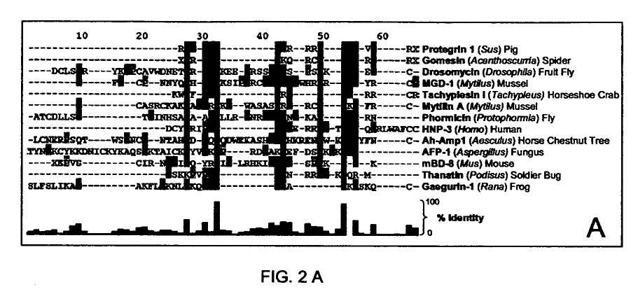

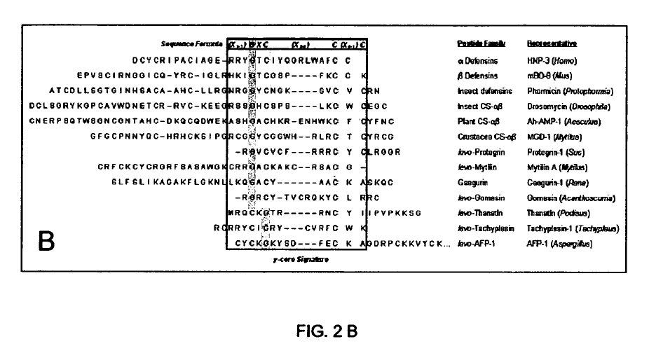

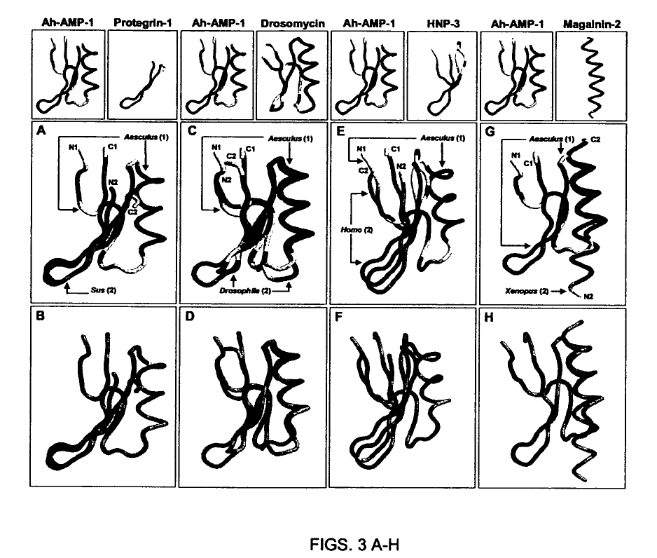

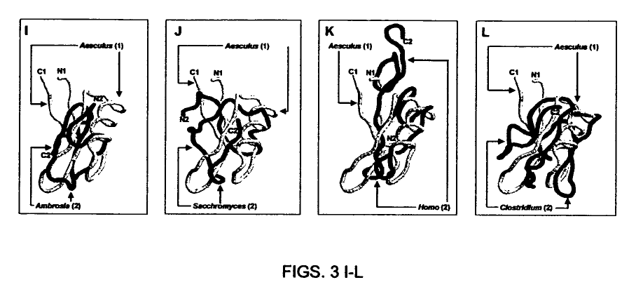

The γ-core motif is a pivotal element in the multidimensional signature of antimicrobial peptides. This motif corresponds to a hydrophobic and structurally rigid region in these molecules. Moreover, the γ-core motif consists of hallmark amino acid sequence, composition, and distribution patterns that likely facilitate antimicrobial functions. For example, patterns identified are congruent with segregation of the most polar or charged residues to solvent-accessible facets, continuity of hydrophilic or hydrophobic surfaces, and flexibility near structural extremities of these peptides. Such physicochemical properties appear to be integral to the antimicrobial mechanisms of disulfide-containing peptides such as the CS-αβ or defensin families (Yeaman and Yount, Pharmacol. Rev. 55:27 (2003); Hill et al., Science 251:1481 (1991)). Thus, the γ-core motif is more than simply a β-hairpin fold. As described herein, the γ-core component of the antimicrobial peptide signature can be derived from dextromeric or levomeric sequence patterns (FIG. 2B). The necessity for host defense against microbial pathogens has favored conservation of an effective 3-dimensional determinant, despite site- or orientation-specific variations in the primary sequences that comprise this motif. Thus, the present invention provides a method for stereospecific analysis of primary sequences that can identify structural patterns or relationships in any protein class selected by the user.

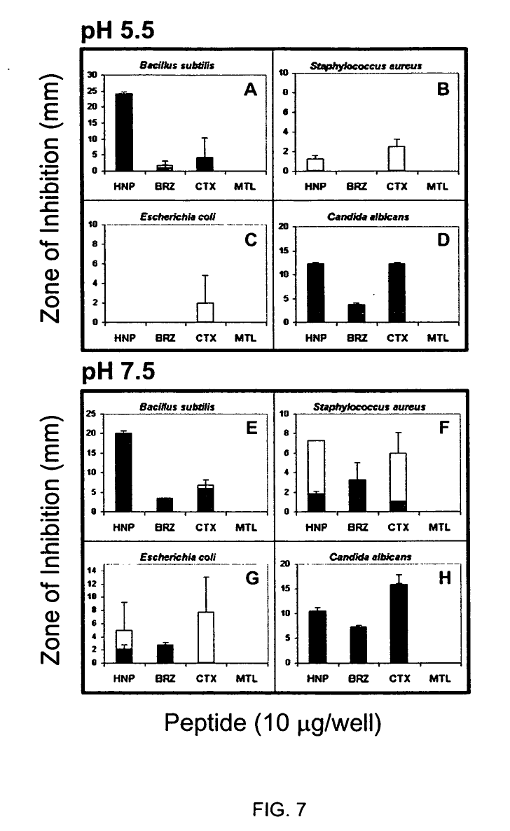

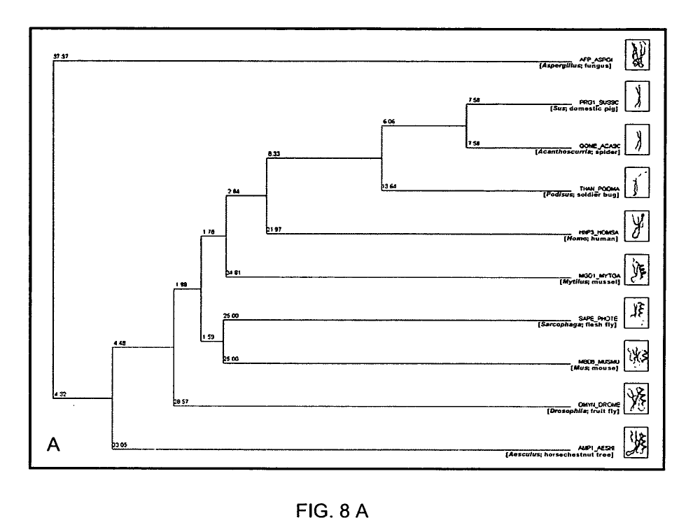

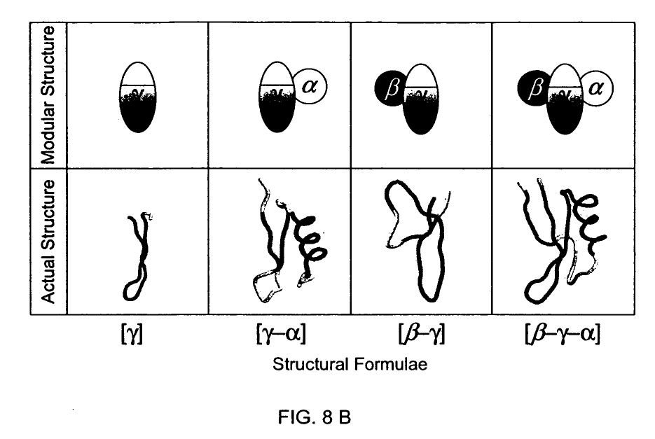

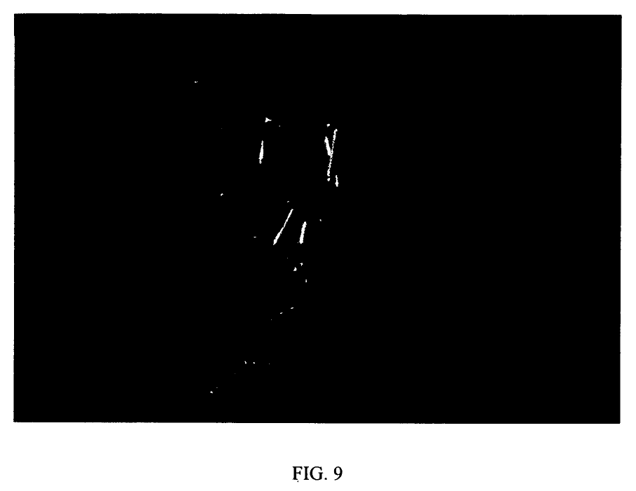

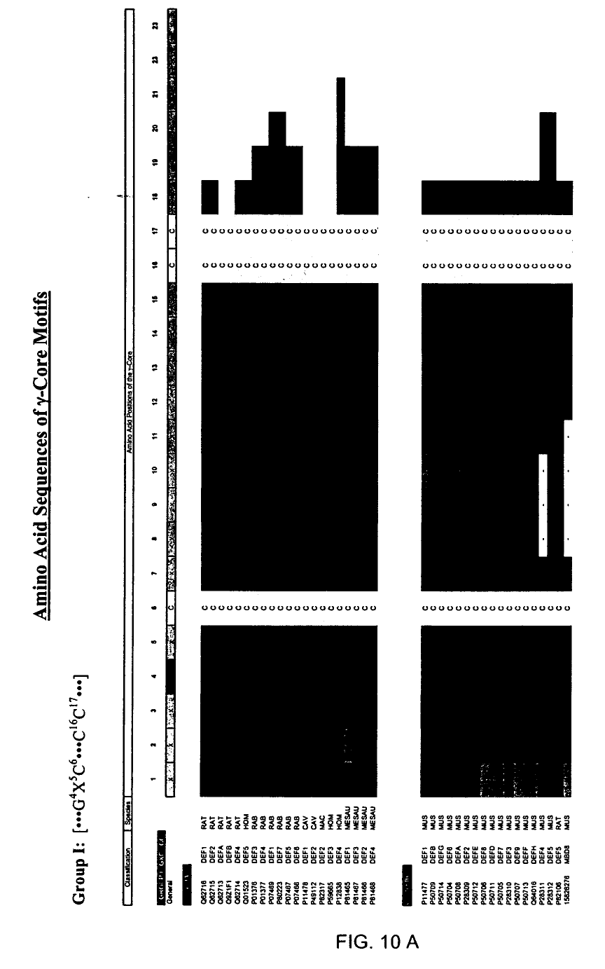

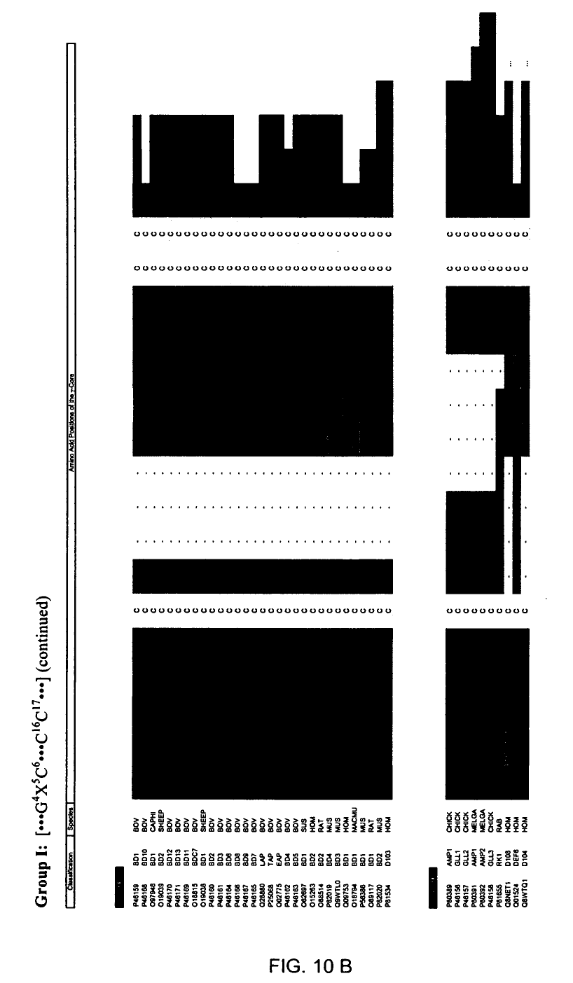

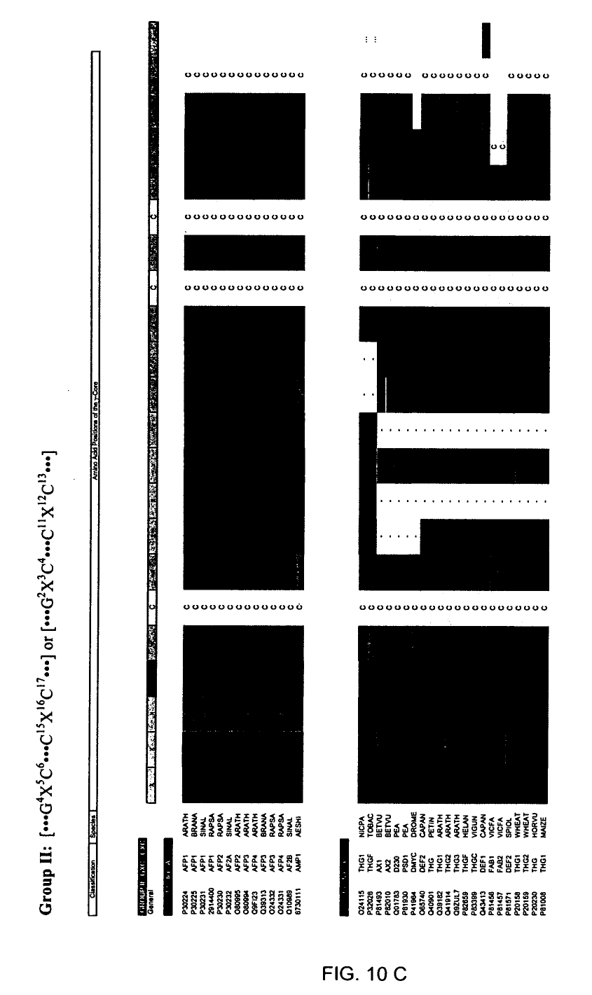

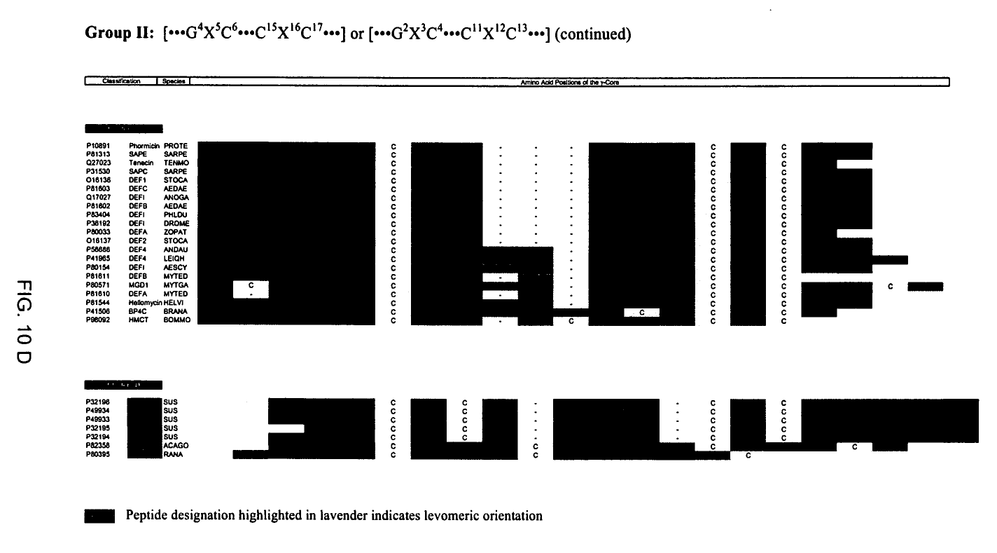

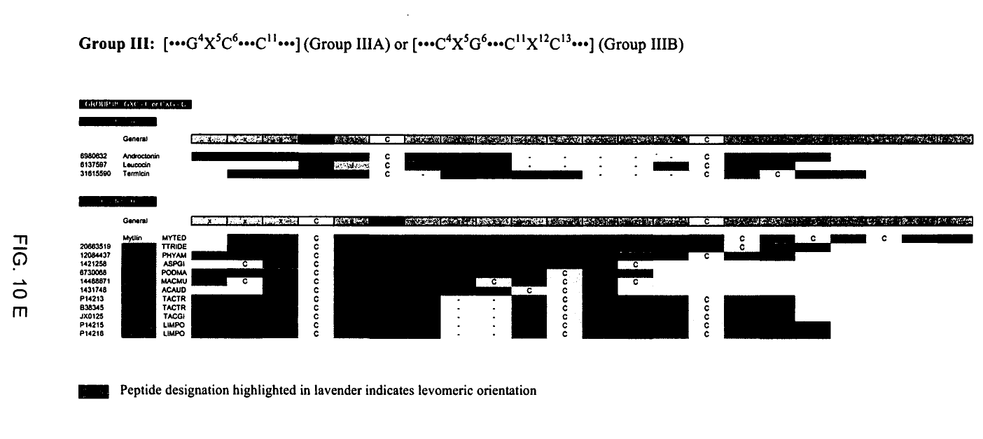

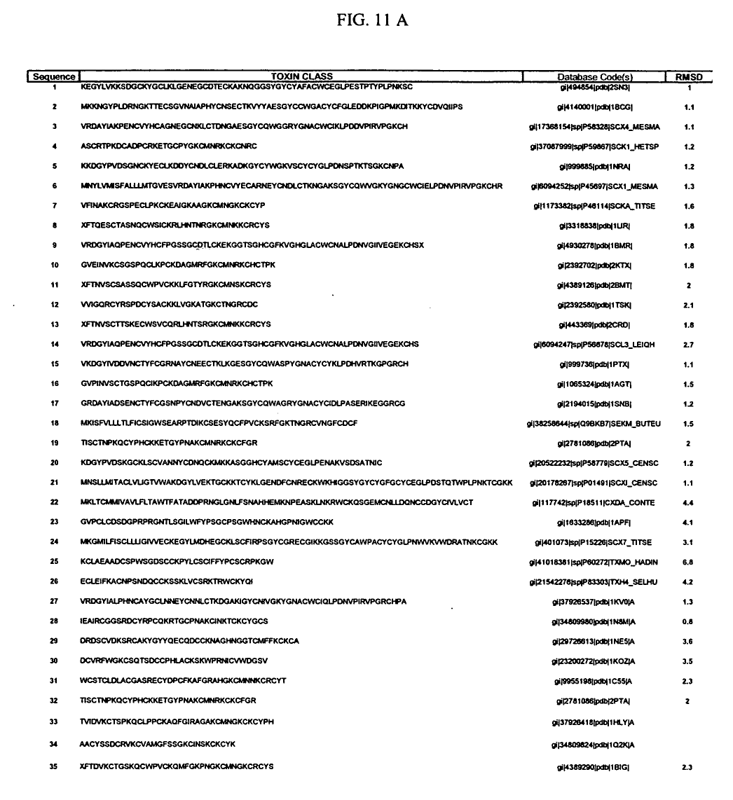

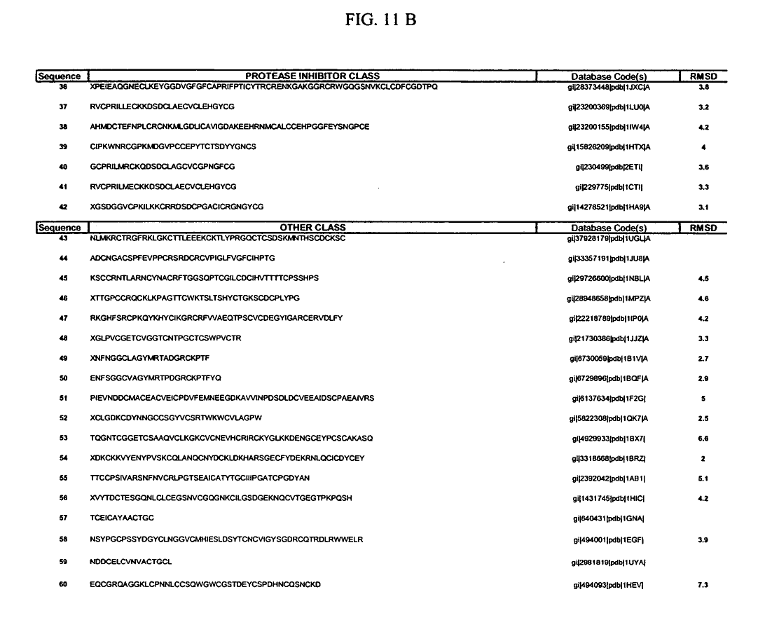

Conservation of the γ-core motif across the phylogenetic spectrum demonstrates it is an archetype of the antimicrobial peptide signature (FIG. 7A). Yet, the γ-core is not necessarily an exclusive structural determinant of antimicrobial activity. In some cases, the γ-core alone is sufficient for antimicrobial activity (eg., protegrins, tachyplesins, RTD-1). However, the motif also can serve as a scaffold, to which complementary antimicrobial determinants (eg., α-helices or β-sheets) are added as adjacent modules.

Thus, disulfide-stabilized antimicrobial peptides represent structural modules coordinated in varying configurations relative to the γ-core (FIG. 7B). Examples of the invention discovery are abundant in nature: Protegrin-1 illustrates the simplest configuration, consisting solely of the γ-core, represented by the modular formula [γ]; MGD-1 contains an α-helical module linked to a γ-core, collectively represented as [γ-α]; alternatively, HNP-3 exemplifies the addition of a β-sheet module to the γ-core, represented as [β-γ]; Ah-AMP-1 illustrates a more complex configuration in which β-sheet and α-helical modules are linked to the γ-core, represented by the formula [β-γ-α]. Permutations of these modular formulae are readily observed in naturally-occurring antimicrobial peptides, encompassing diverse antimicrobial peptide families, including α-defensins, β-defensins, θ-defensins, cathelicidins, protegrins, and CS-αβ peptides found in plants, invertebrates, insects, and arthropods. Based on this discovery the present invention provides methods of utilizing specific mosaic configurations of such structural modules to optimize the function of a given antimicrobial peptide against relevant pathogens in specific physiologic contexts.

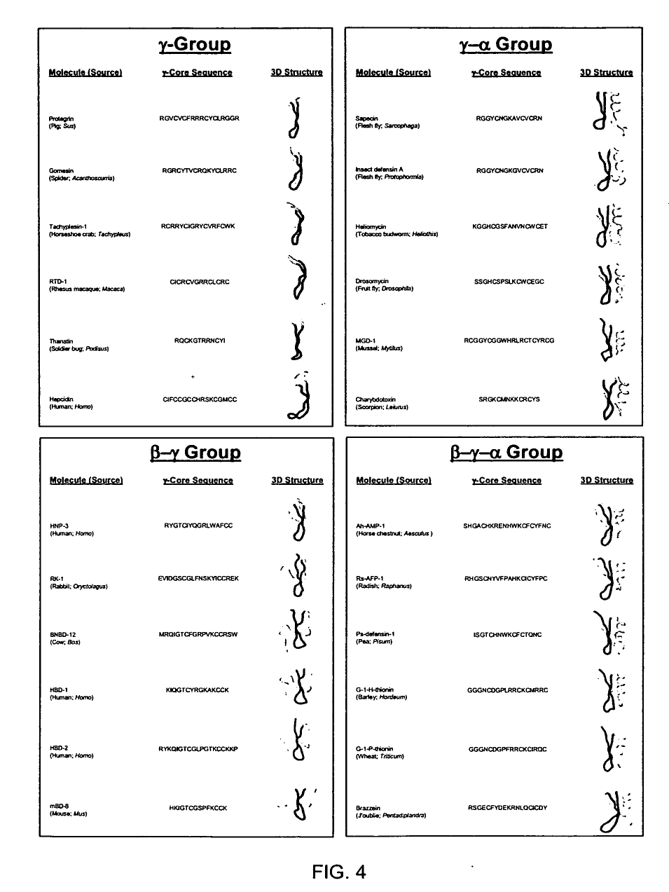

Thus, peptides with common evolutionary precursors may have conserved structural elements independent of functional divergence. As one verification of this discovery, AFP-1 and TGF-β were intentionally included in the exemplified phylogenetic and structural analyses as relative outliers in the comparative antimicrobial and non-antimicrobial peptide groups. This level of divergence is reflected in their significant phylogenetic distances from other peptides in their respective subsets. Yet, as described herein, despite equidistant divergence from Ah-AMP-1, AFP-1 exhibits the fundamental γ-core signature of antimicrobial peptides, while TGF-β does not (FIGS. 3 and 4). This result reinforces the importance of the γ-core motif as part of a multidimensional signature for antimicrobial activity. Moreover, structural divergence of AFP-1 from other antimicrobial peptides lies predominantly in modules beyond the γ-core. Thus, as exemplified for AFP-1, the invention provides new insights into eukaryotic evolution of the multidimensional signature of antimicrobial peptides that confer survival advantages in environments rich in microbial pathogens.

The discovery of a multidimensional signature as described herein can be applied to a method of identifying peptides that exert previously unrecognized antimicrobial activity. As described herein, for example, the sweetener protein, brazzein, and the scorpion neurotoxin, charybdotoxin, were found to have previously unrecognized antimicrobial activity against bacteria and fungi. The present model also accurately predicted that the prototype metallothionein II, which fulfilled the primary sequence pattern, but lacked the 3-dimensional criteria of the antimicrobial signature, was devoid of antimicrobial activity. As described herein, the multidimensional signature model was further substantiated by successful prediction of the γ-core motif in tachyplesins of unknown 3-dimensional strucuture, but which had known antimicrobial activity, and fulfilled the primary structure criteria of the model. Together, these findings validate the predictive accuracy, utility and applicability of the multidimensional antimicrobial peptide signature model to the methods provided by the present invention.

As disclosed herein, the multidimensional signature is a unifying structural code for broad classes of host defense peptides. This discovery is supported, for example, in the exemplification that a major class of peptides can be retrieved from the protein database searches using the stereospecific sequence formulae consisting of protease inhibitors and related proteins derived from plants (Table II). The botanical and related literature indicate that several such peptides have been shown to be plant defensins (Sallenave, Biochem. Soc. Trans. 30:111 (2002); Wijaya et al., Plant Sci 159:243 (2000)). Moreover, the plant proteinase inhibitor superfamily includes thionin peptides containing the antimicrobial γ-core motif as disclosed herein (Table I; Melo et al., Proteins 48:311 (2002)). In addition, peptides originally identified as having cytokine bioactivities are now known to have direct antimicrobial activity. Examples include γ-chemokines such as human platelet factor-4 and platelet basic peptide (PF-4 and PBP; Tang et al., Infect. Immun. 70:6524 (2002); Yeaman, Clin. Infect. Dis. 25:951 (1997)), monokine induced by interferon-y (MIG/CXCL9; Cole et al., J. Immunol. 167:623 (2001)), interferon-γ inducible protein-10 kDa (IP-10/CXCL10; Cole et al., J. Immunol. 167:623 (2001)), interferon-inducible T cell α chemoattractant (ITAC/CXCL11; Cole et al., J. Immunol. 167:623 (2001)), and the β-chemokine, RANTES (releasable upon activation normal T cell expressed/secreted; Tang et al., Infect. Immun. 70:6524 (2002); Yeaman, Clin. Infect. Dis. 25:951 (1997)). Importantly, each of these proteins contains an iteration of the multidimensional antimicrobial signature as provided by the present invention. Collectively, these observations demonstrate the link between the multidimensional antimicrobial signature, and functional correlates in multifunctional host defense peptides (Yeaman, Clin. Infect. Dis. 25:951 (1997); Ganz, Science 298:977 (2002)). The skilled person will appreciate that the multidimensional antimicrobial signature can be found in additional peptides, and that the presence of this signature is associated with antimicrobial activity.

Multidimensional signatures of antimicrobial peptides exemplify how nature can diverge at the level of overall amino acid sequence, yet preserve essential primary sequence patterns and 3-dimensional determinants effective in host defense. Thus, critical structures of antimicrobial peptides from evolutionarily distant organisms such as microbes and plants are recapitulated in higher organisms, including humans. As disclosed herein, vertical and horizontal acquisition of genes, along with their recombination, yield mosaic iterations upon key structural determinants, such as the γ-core motif (Bevins et al., Genomics 31:95 (1996); Gudmundsson, et al., Proc. Natl. Acad. Sci. USA 92:7085 (1995)). Selective pressures favoring this remarkable degree of structural conservation can include genetic selection against structural variants, and convergent evolution of independent ancestral templates. It follows that the γ-core signature is incorporated into a variety of structural mosaics (eg., [γα₁], [γβ₁], or [γα₁β₁]) readily observed amongst disulfide-stabilized antimicrobial peptides along the phylogenetic spectrum. While future studies will resolve their precise phylogenetic lineage, the multidimensional signatures in antimicrobial peptides likely reflect fundamental host-pathogen interactions and their co-evolution.

The discovery and characterization of antimicrobial peptide signatures can also provide insights for development of new generation anti-infective agents. For example, most microbial pathogens are unable to acquire rapid or high-level resistance to antimicrobial peptides. Critical structure-activity relationships in these molecules can circumvent microbial resistance mechanisms, and interfere with essential microbial targets distinct from classical antibiotics (Yeaman and Yount, Pharmacol. Rev. 55:27 (2003)). Such modes of action exploit pathogen-specific structures intrinsically difficult to mutate, limiting the development of resistance through target or pathway modification. Thus, structural signatures in antimicrobial peptides can advance the discovery and development of improved anti-infective agents and strategies that are refractory to microbial resistance. Therefore, the invention provides a method of improving the antimicrobial activity of a protein by altering the multidimensional signature. Methods of protein design are well known in the art as described, for example, in Concepts in Protein Engineering and Design: An Introduction; Wrede and Schneider (Eds.), Walter de Gruyter, Inc. (pub.), 1994); Evolutionary Approaches to Protein Design, Vol. 55, Frances H. Arnold (Ed.), Edward M. Scolnick (Ed.), Elsevier Science & Technology Books, 2000; Molecular Design and Modeling: Concepts and Applications, Part A: Proteins, Peptides, and Enzymes: Volume 202: Molecular Design and Modelling Part A, John N. Abelson (Ed.), John J. Langone (Ed.), Melvin I. Simon (Ed.), Elsevier Science & Technology Books, 1991; and Protein Engineering and Design, Paul R. Carey (Ed.), Elsevier Science & Technology Books, 1996; all of which are incorporated herein by reference in their entirety.

It is understood that modifications which do not substantially affect the activity of the various embodiments of this invention are also included within the definition of the invention provided herein. Accordingly, the following examples are intended to illustrate but not limit the present invention.

### EXAMPLE I

### Identification of Multidimensional Signatures of Antimicrobial Peptides

This Example shows identification of a disulfide-stabilized core motif that is integral to the 3-dimensional signature of cysteine-containing antimicrobial peptides.

The relatedness amongst primary structures was examined in prototypic cysteine-containing antimicrobial peptide sequences representing taxa spanning an evolutionary distance of 2.6 billion years (BY; estimated date of phylogenetic divergence of fungi and plants from higher organisms; Nei et al., Proc. Natl. Acad. Sci. U S A. 98:2497 (2001)). A prototype from each class of non-cyclic, disulfide-containing antimicrobial peptides was represented in these analyses [Antimicrobial peptides were selected from the National Center for Biotechnology Information (NCBI) Entrez Protein (www.ncbi.nlm.nih.gov:80/entrez/) or Antimicrobial Sequences (www.bbcm.univ.trieste.it/˜tossi/) databases.]

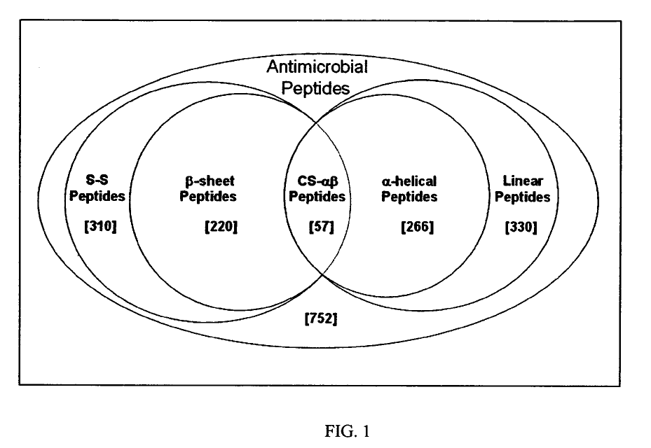

The specific criteria for selection of peptides analyzed included: 1) eukaryotic origin; 2) published antimicrobial activity; 3) non-enzymatic mechanism(s) of action; 4) mature protein sequence; and 5) less than 75 amino acids in length. Peptides for which structures have been determined were used in structural analyses [Peptides were selected from the National Center for Biotechnology Information (NCBI) structure (www.ncbi.nlm.nih.gov:80/entrez/) and Protein Data Bank (PDB) (www.rcsb.org/pdb/) resources.]. The resulting study set included antimicrobial peptides encompassing a broad distribution in source (i.e., biological kingdoms ranging from microorganisms to man), amino acid sequence, and conformation class (FIG. 1). Amino acid sequence data were used for these analyses, as not all nucleotide sequences have been characterized, and saturation of nucleotide sequence data occurs within non-mitochondrial sequences over evolutionary timescales.

FIG. 1 shows conventional antimicrobial peptide structure classification and distribution. The relationship amongst structure and predominance is summarized for the commonly recognized antimicrobial peptide classes. Concatenation represents the proportionate distribution of peptides encompassing a given structural class, as calculated from the Antimicrobial Sequences Database. Antimicrobial peptides were selected from the National Center for Biotechnology Information (NCBI) Entrez Protein (www.ncbi.nlm.nih.gov:80/entrez/) or Antimicrobial Sequences (www.bbcm.univ.trieste.it/˜tossi/) databases. The numbers of peptides classified in each group are indicated in brackets for each class. Of the more than 750 peptides present in the database at the onset of the study, the balance of those not indicated are comprised of peptides representing unusual or other classifications, including macrocyclic, proline-rich, tryptophan-rich or indolicidin-like peptides, and large polypeptides greater than 75 amino acids in length.

Representatives included antimicrobial peptides from taxa encompassing broad biological diversity spanning an evolutionary distance of 2.6 billion years (estimated divergence of fungi and plants from higher organisms; [Nei et al, Proc. Natl. Acad. Sci. USA 98:2497 (2001).]). This dataset included prototypes of all major classes of disulfide-containing antimicrobial peptides, including distinct conformation groups such as defensin, cysteine-stabilized αβ, ranabox and β-hairpin.

Conventional MSA (N to C terminal; dextromeric) revealed no clear consensus patterns amongst primary sequences of the antimicrobial peptide study set. However, visual inspection revealed an absolutely conserved GXC motif, oriented in reverse in some peptides. We hypothesized that conventional MSA failed to recognize this inverted consensus pattern. Therefore, peptides containing inverted GXC motifs were aligned in their C to N terminal (levomeric) orientation. This stereospecific MSA revealed a novel and striking sequence pattern common to all disulfide-containing antimicrobial peptide classes (Figure X). The consensus patterns, defined herein as the enantiomeric sequence signature, adhere to the formulae:
NH₂ . . . [X₁₋₃]-[GXC]-[X₃₋₉]-[C] . . . COOH (dextromeric isoform)
NH₂ . . . [C]-[X₃₋₉]-[CXG]-[X₁₋₃] . . . COOH (levomeric isoform 1)
NH₂ . . . [C]-[X₃₋₉]-[GXC]-[X₁₋₃] . . . COOH (levomeric isoform 2)

These consensus patterns transcend defensin-specific motifs identified previously (White et al., Curr. Opin. Struct. Biol. 5:521 (1995); Yount et al., J. Biol. Chem. 274:26249 (1999). Specific characteristics of the enantiomeric sequence signatures include: i) a length of 8-16 amino acid residues; and ii) conserved GXC or CXG motifs within the sequence isoforms. Interestingly, levomeric isoform 2 peptides retain a dextromeric GXC motif within the levomeric sequence signature (Figure x).

Identification of the conserved enantiomeric signature suggested that a corresponding motif would also be present in the 3-dimensional structures of disulfide-stabilized antimicrobial peptides. Conformation alignments revealed a core motif that was absolutely conserved across all classes of disulfide-stabilized antimicrobial peptides (Figure x; Table x). This 3-dimensional archetype, termed herein as the γ-core motif, is comprised of two anti-parallel β-sheets, interposed by a short turn region (Figures x and x). All three isoforms of the enantiomeric sequence signature conform to the γ-core motif, reflecting their 3-dimensional convergence (Figure x). Additional features that characterize the γ-core include: 1) net cationic charge (+0.5 to +7) with basic residues typically polarized along its axis; 2) periodic charge and hydrophobicity yielding amphipathic stereogeometry; and 3) participation in 1-4 disulfide bonds. This motif may comprise the entire peptide, or link to adjacent structural domains.

Relative to the γ-core, disulfide-stabilized antimicrobial peptides of evolutionarily distant organisms exhibited a striking convergence in conformation, that was essentially isomeric, or at a minimum, highly homologous (Figure x). This 3-dimensional convergence encompassed overall conformations, or localized to specific domains in comparative peptides. For example, the structures of Ah-AMP-1 (horsechestnut tree, Aesculus) and drosomycin (fruit fly, Drosophila) are essentially superimposable over their entire backbone trajectories (Figure x). Alternatively, protegrin-1 (domestic pig, Sus) and Ah-AMP-1 share conformational homology corresponding to their γ-core motifs (Figure x). As anticipated, magainin aligned to the α-helical motif in Ah-AMP-1 (Figure x), verifying the specificity of conformational alignments.

To confirm the significance of 3-dimensional convergence in the antimicrobial peptide signature, comparisons between representative cysteine-containing antimicrobial and non-antimicrobial peptides of equivalent molecular weight were performed and analyzed. Outcomes emphasize that non-antimicrobial peptides fail to achieve the multidimensional signature of antimicrobial peptides (FIG. 3I-L). Mean quantitative RMSD confirmed the statistical significance of the differences between antimicrobial and non-antimicrobial structures (Table I).

TABLE I
Quantitative analysis of 3-dimensional convergence amongst prototypic
antimicrobial peptide structures.

| | AAs | RMSD (Å) | Identity (%) | Align/Gap |
|---|---|---|---|---|
| Antimicrobial Peptides | | | | |
| Ah-AMP-1 (Aesculus; Tree; 1BK8; 28) | 50 | 0.0 | 100 | 50/0 |
| Sapecin (Sarcophaga; Fly; 1L4V; 29) | 40 | 0.9 | 25.0 | 38/0 |
| Protegrin-1 (Sus; Pig; 1PG1; Fahrner et al., Chem. | 19 | 1.2 | 18.8 | 16/0 |
| Biol. 3: 543 (1996)) | | | | |
| Drosomycin (Drosophila; Fruit Fly; 1MYN; | 44 | 1.4 | 29.3 | 41/6 |
| Landon et al., Protein Sci. 6: 1878 (1997)) | | | | |
| Defensin (Raphanus; Radish; 1AYJ; Fant et al., J. | 51 | 1.3 | 47.6 | 49/0 |
| Mol. Biol. 279: 257 (1998)) | | | | |
| Thionin (Triticalis; Wheat; 1GPS; Bruix et al., | 47 | 1.8 | 26.1 | 46/3 |
| Biochemistry 32: 715 (1993)) | | | | |
| MGD-1 (Mytilus; Mussel; 1FJN; Yang et al., | 39 | 2.0 | 26.5 | 34/1 |
| Biochemistry 39: 14436 (2000)) | | | | |
| Thanatin (Podisus; Soldier Bug; 8TFV; Mandard et | 21 | 2.2 | 12.5 | 16/0 |
| al., Eur. J. Biochem. 256: 404 (1998)) | | | | |
| HNP-3 (Homo; Human; 1DFN; Hill et al., Science | 34 | 3.2 | 8.3 | 24/17 |
| 251: 1481 (1991)) | | | | |
| MBD-8 (Mus; Mouse; 1E4R; Bauer et al., Protein | 35 | 3.4 | 0.0 | 24/13 |
| Sci. 10: 2470 (2001)) | | | | |
| AFP-1 (Aspergillus; Fungus; 1AFP; Campos-Olivas | 51 | 4.8 | 6.2 | 32/7 |
| et al., Biochemistry 34: 3009 (1995)) | | | | |
| Mean ± SD | | 2.2 ± 1.2* | | |
| Non-Antimicrobial Peptides | | | | |
| TGF-α (Homo; Human; 3TGF; Harvey et al., Eur. | 50 | 4.7 | 3.1 | 32/7 |
| J. Biochem. 198: 555 (1991)) | | | | |
| Metallothionein (Saccharomyces; Yeast; 1AOO; | 40 | 5.3 | 18.8 | 32/16 |
| Peterson et al., FEBS Lett. 379: 85 (1996)) | | | | |
| Allergen-5 (Ambrosia; Ragweed; 2BBG; Metzler et | 40 | 6.5 | 18.8 | 32/7 |
| al., Biochemistry 31: 5117 (1992)) | | | | |
| Ferredoxin (Clostridium; Bacteria; 2FDN; Dauter et | 55 | 7.4 | 5.0 | 40/6 |
| al., Biochemistry 36: 16065 (1997)) | | | | |
| Mean ± SD | | 6.0 ± 1.2* | | |

Briefly, three-dimensional alignments of representative antimicrobial and control non-antimicrobial peptide structures were analyzed by pairwise comparison with Ah-AMP-1 (Aesculus; horsechestnut tree; 1BK8) using the combinatorial extension method (Shindyalov and Bourne, Protein Eng. 11:739 (1998)). Control peptides were selected from a cohort of 54 appropriate comparators based on disulfide content, sequence length, and molecular weight equivalence to Ah-AMP-1. Representative results are shown. The comparative length of each mature peptide is indicated as the number of amino acids (AAs). Root Mean Square Deviation (RMSD) values were determined for distances between α-carbon atoms over the length of the alignment. Percent identity is the percentage of sequence identity between the two peptides compared. The align/gap value indicates the number of residues considered for the alignment, and the number of gaps inserted. Relative gap penalties were integrated into the analysis. Mean RMSD values from antimicrobial versus non-antimicrobial peptides were significantly different (*) as determined by two tailed T-test (P <0.01). Information for each structure is formatted as follows: peptide name, (source genus; common name; Protein Data Bank [PDB] accession code; reference).

A highly conserved, disulfide-stabilized core motif was discovered to be integral to the 3-dimensional signature of cysteine-containing antimicrobial peptides. This feature is termed herein as the gamma-core motif (γ-core; FIG. 4A). This structural motif is comprised of two anti-parallel β-sheets interposed by a short turn region. Notably, as shown in FIG. 4, the sequence patterns corresponding to the γ-core signature extends across the entire range of antimicrobial peptide families. Exemplary peptides incuded within the groups are: gomesin ([1KFP], Acanthoscurria, spider, (γ-Group); Mandard et al., Eur. J. Biochem. 269:1190 (2002)); protegrin-1 ([1PG1], Sus, domestic pig, (γ-Group)); thanatin ([8TFV], Podisus, soldier bug, (γ-Group)); α-defensin (HNP-3, [1DFN]; Homo, human, (β-γ-Group); β-defensin (MBD-8, [1E4R], Mus, mouse, (β-γ-Group)); fungal peptide (AFP-1, [1AFP], Aspergillus, fungus, (β-γ-α Group); insect-defensin (sapecin, [1L4V], Sarcophaga, flesh fly, (γ-α-Group)); crustacean

CS-αβ peptide (MGD-1, [1FJN], Mytilus, mussel, (γ-α-Group)); insect CS-αβ peptide (drosomycin, [1MYN], Drosophila, fruit fly, (γ-α-Group)); and plant CS-αβ peptide (Ah-AMP-1, [1BK8] Aesculus, horsechestnut tree, (β-γ-α Group). Other peptide data are formatted as in FIG. 3. See Table I for additional references. The conserved GXC (dextromeric) or CXG (levomeric) sequence patterns (FIG. 2B) are integrated into one β-sheet in this motif, reflecting conformational symmetry amongst antimicrobial peptides containing this signature (FIG. 4B, respectively). Additional features that distinguish the γ-core include: 1) hydrophobic bias toward the C-terminal aspect; and 2) cationic charge positioned at the inflection point and termini of the β-sheet domains, polarizing charge along the longitudinal axis of the γ-core.

### EXAMPLE II

### Validation of the Multidimensional Antimicrobial Peptide Signature Model

The multidimensional signature model for antimicrobial peptides integrates a stereospecific (dextromeric or levomeric) sequence pattern with the 3-dimensional gamma-core (“γ-core”). Therefore, this model predicted that peptides fulfilling these prerequisites would exert antimicrobial activity, even though such activity may not yet have been determined. Multiple and complementary approaches were used to test the model in this regard: 1) prediction of antimicrobial activity in peptides fulfilling the sequence and conformation criteria of the multidimensional signature, but not yet recognized to have antimicrobial activity; 2) predicted failure of antimicrobial activity in peptides exhibiting primary sequence criteria, but lacking the 3-dimensional γ-core signature of the model; and 3) prediction of a γ-core motif in disulfide-containing peptides with known antimicrobial activity, and which fulfilled primary sequence criteria, but had unknown structure.

To test the hypothesis that the primary sequence patterns of the multidimensional signature are relevant to all classes of disulfide-containing antimicrobial peptides, Swiss-Prot forward and reverse databases (Gattiker et al., Appl. Bioinformatics 1:107 (2002)) were queried with the enantiomeric sequence formulae. Representatives of all major disulfide-containing antimicrobial peptide classes were retrieved (Table II). Searches also retrieved members of other peptide subclasses: i) neurotoxins, particularly charybdotoxin class of the family Buthidae (scorpion); ii) protease inhibitor or related peptides (eg., brazzein) from plants; iii) ferredoxins; and iv) metallothioneins. Prototypes with known 3-dimensional structures, but no known antimicrobial activity, were analyzed for the presence of the γ-core signature. Of these, the peptides brazzein and charybdotoxin were selected to test for antimicrobial activity based on two criteria: i) their quantitative RMSD values reflected greatest homology to the comparator 7-core motif; and ii) they represented diverse non-mammalian (plant or scorpion) host sources and distinct structure classes not previously known to have antimicrobial activity. Thus, brazzein and charybdotoxin exemplified peptides that fulfilled the enantiomeric sequence and γ-core criteria required for the multidimensional signature. These peptides were predicted to have direct antimicrobial activity. In contrast, prototype metallothioneins and ferredoxins did not contain γ-core motifs (FIG. 3; Table II). Thus, metallothionein II was selected as an example comparator predicted to lack antimicrobial activity.

| | | Sequence Isoform | | | Proportion | |
|---|---|---|---|---|---|---|
| Antimicrobial Peptide Class | Phylogeny | Dextro | Levo - 1 | Levo - 2 | Total | % Total |
| α-defensin | Chordata | 24 | 42 | 6 | 72 | 15.3 |
| β-defensin | Chordata | 52 | 65 | 31 | 148 | 31.4 |
| θ-defensin | Chordata | 1 | 1 | 0 | 2 | 0.4 |
| Insect defensin/CS-αβ | Insectae | 21 | 23 | 12 | 56 | 11.9 |
| Plant defensin/CS-αβ | Plantae | 51 | 67 | 20 | 138 | 29.3 |
| Invertebrate defensin/CS-αβ | Mollusca | 3 | 4 | 4 | 11 | 2.3 |
| Protegrins/Gomesins | Chordata/Arthropoda | 0 | 0 | 6 | 6 | 1.3 |
| Tachyplesins/Polyphemusins | Arthropoda | 6 | 5 | 2 | 13 | 2.8 |
| Thanatin | Arthropoda | 0 | 1 | 0 | 1 | 0.2 |
| Mytilins/Big-Defensin | Mollusca | 3 | 3 | 2 | 8 | 1.7 |
| AFP-1 | Ascomycota | 1 | 0 | 0 | 1 | 0.2 |
| Lantibiotics/Microcins | Proteobacteria | 3 | 3 | 9 | 15 | 3.2 |
| | | 165 | 214 | 92 | 471 | |

Table II. Recognition of diverse classes of antimicrobial peptides by the enantiomeric sequence formulae. Forward or reverse Swiss-Prot Databases (release 42.4; Nov. 14, 2003; 138,347 entries) were probed with formulae containing the dextromeric or levomeric motifs of the antimicrobial peptide signature using PROSITE (Gattiker et al., Apl. Bioinformatics 1:107 (2002)). Data indicate the proportionate distribution of a non-redundant cohort of retrieval sets; in some cases, peptides were retrieved by more than one formula isoform. Note that search results include members of the lantibiotic superfamily of antimicrobial peptides that lack conventional disulfide bridges, but have alternate thioether stabilization.

These peptides were tested for antimicrobial activity against a panel of Gram-positive (Staphylococcus aureus, Bacillus subtilis) and Gram-negative (Escherichia coli) bacteria, and the fungus Candida albicans, using a well-established and sensitive in vitro assay [Antimicrobial activity was assessed using a well-established solid-phase diffusion method. Assays included well-characterized organisms: Staphylococcus aureus (ATCC 27217, Gram-positive); Bacillus subtilis (ATCC 6633, Gram-positive); Escherichia coli (strain ML-35, Gram-negative); and Candida albicans (ATCC 36082, fungus). In brief, organisms were cultured to logarithmic phase and inoculated at a density of 10⁶ colony forming units/ml in buffered molecular grade agarose at the indicated pH. Five μg of peptide resuspended in sterile deionized water were introduced into wells formed in the underlay, and incubated for 3 h at 37° C. Nutrient-containing overlay medium was then applied, and assays incubated at 37° C. or 30° C. for bacteria or fungi, respectively. Defensin HNP-1 was tested in parallel as a standard control. After 24 h, zones of complete or partial inhibition were measured. All assays were repeated independently a minimum of two times. Tang et al., Infect. Immun. 70:6524 (2002) for detailed methodology.].

As predicted by the signature model, brazzein and charybdotoxin exerted direct antimicrobial activity against bacteria and C. albicans (FIG. 7). Notably, these peptides exhibited pH-specific antimicrobial activities, which in some conditions exceeded that of HNP-1. These results demonstrate for the first time to our knowledge the direct antimicrobial activities of brazzein and charybdotoxin. In contrast, metallothionein II failed to exert antimicrobial activity against any organism tested under any condition, as predicted by the model.

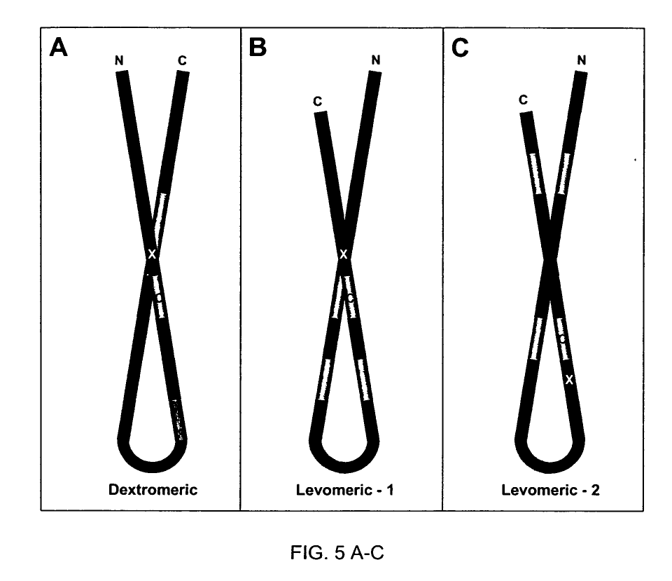

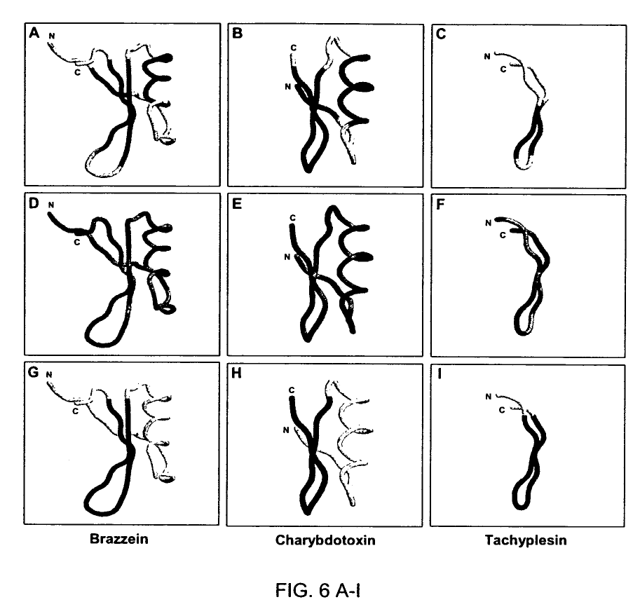

An alternative approach was also used to validate the multidimensional signature model. Tachyplesins are known cysteine-containing antimicrobial peptides from the horseshoe crab, Tachypleus. Two tachyplesins were retrieved from protein database searches employing the levomeric sequence formula (Table II). The model predicted that, because they have known antimicrobial activity, and fulfill the primary sequence criteria, tachyplesins would contain a γ-core motif. The 3-dimensional structure of tachyplesin I became available subsequent to development of the model (Laederach et al., Biochem. 41:12359 (2002)), and as predicted, exhibits a γ-core motif integral to the multidimensional signature of disulfide-containing antimicrobial peptides (FIG. 5). Confirmation of the 3-dimensional γ-core structure from antimicrobial activity and primary sequence pattern offers a robust and complementary validation of the multidimensional signature model.

The phylogenetic relationships among antimicrobial peptides containing the multidimensional signature were also examined. Study peptides sorted in a continuum of increasing structural complexity relative to the γ-core motif, rather than evolutionary relatedness of the source organisms (FIG. 7A). This phylogenetic pattern is consistent with conservation of the γ-core motif amongst cysteine-containing antimicrobial peptides across biological kingdoms.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1. A method for predicting antimicrobial activity of a candidate protein comprising determining the the presence a multidimensional antimicrobial signature in a candidate protein, and comparing said multidimensional antimicrobial signature to a multidimensional antimicrobial signature model, whereby the degree of similarity between the multidimensional antimicrobial signature to the multidimensional antimicrobial signature model is predictive of antimicrobial activity.

2. A method for identifying a protein having antimicrobial activity comprising screening a library of candidate proteins to identify a multidimensional antimicrobial signature in a candidate protein, and comparing said multidimensional antimicrobial signature to a multidimensional antimicrobial signature model, whereby the degree of similarity between the multidimensional antimicrobial signature to the multidimensional antimicrobial signature model is predictive of antimicrobial activity.

3. A method for improving the antimicrobial activity of a protein comprising altering the multidimensional antimicrobial signature of the protein to increase the degree of similarity between said multidimensional antimicrobial signature and a multidimensional antimicrobial signature model, thereby improving the antimicrobial activity of the protein.

4. A method for designing a protein having antimicrobial activity comprising incorporation of configurations comprising iterations of a γ-core signature in a peptide structure.
